(19) United States
(12) Patent Application Publication
(10) Pub. No.: US 2009/0246298 A1
(43) Pub. Date: Oct. 1, 2009

(54) Viagra

(75) Inventor: Ceola Pattiee Steele, Newark, NJ (US)

Correspondence Address: CEOLA P. STEELE, P.O. Box 22409, NEWARK, NJ, 07101, US

(73) Assignee: CEOLA P. STEELE

(21) Appl. No.: 12/009,258

(22) Filed: Mar. 31, 2008

(51) Int. Cl. A61K 36/00; A61P 15/10

(52) U.S. Cl. 424/725

(57) ABSTRACT

1. Couch Grass works on the prostate gland to clear it of bacteria. If this bacteria is allowed to build up it could cause arthritis neuralgia, cancer, and neurological, problems. The blood is cleaned by the Magistral Viagra and hormones are activated. Couch Grass is in the carrot family, it is potent. The potency of Couch Grass is 10 to 1 it is 10× more potent than carrots. It is very high in potassium.
2. Stale Red Clover, quantity of herb to be used is, judged on the weight of 150 pound man. This herb contains ammonia fixing bacteria, it works on the kidneys. The sodium, potassium cells of the kidneys are activated. Salts and potassium cells of the kidneys are activated. Salts and potassium work on the gut to produce a super abundant amount of energy, water is the catalyst for the production of hormones in the blood. These hormones travel through out the body in the blood. They work on the principle of vasodilation and vasoconstriction. I want to focus on the vasoconstriction.
3. Skull Cap works on the body using the volatile fixed oils, (essential oil) The main color titrated out is green, green works on the heart. In the Contraction phase the heart rests. There are seven botanical herbs in the Magistral Viagra. The synergistic effect causes the free flow of hormones in Endocrine system, (duct-less glands) to operate efficiently. It helps to rid the body of diseased bacteria and gives the patient confidence in coitus.

### INGREDIENTS

Couch Grass

Common Names: Dog Grass, Quick Grass, Durfa Grass, Twitch Grass, Twitch Grass And Field of Fire

Botanical Name: Triticum repens or Agropyrum repens, Beav.

### NO.: GRAMINEAE

I used this because of its ability to restore health to the bladder and the urinary tract, by dissolving small calculi, it is a general catarrhal treatment, which if not eliminated advances more seriously. This herb is also good for rheumatic and jaundice suffers. “The accepted administration is an in-fusion of 1 oz. of Couch Grass to 1 pint of boiling water, steeped 20 min. and given in wineglassful doses every 2 hr. or a decoction of the root made by boiling 1 oz of the herb in distilled water until reduced to ¾ pint, 10 to 20 drops in water two or more times a day.” Pg 97, 98 Indian Herbalogy.

This herb is from the carrot family. In my thought process it would activate the cell by the sodium potassium gates, and markedly increase peristalsis. Jet noun 1. A jet-propelled vehicle a high-speed stream forced under pressure out of a small-diameter-opening or nozzle. 2. A jet-propelled vehicle a high-speed stream, E. Equilibrium occurs when the separate distinct parts come together to act on the momentary displacement function of ejecting unwanted bacteria, achieving the fluid stream that you can see.

Thread a noun 1a. Fine cord of fibrous material made of two or more filaments twisted together. b. To make one's way cautiously: through something.

The subjective part, the part you can't see can be worked out in mathematics by, MOMENTUM 2a. The force of motion called “Linier Momentum”: Physics abr. M. The product of a quantity and its perpendicular distance from a reference point Thus, you get a quantum jump. Physics 1. The transition of an atomic or molecular system from one discrete energy level to another with concomitant absorption or emission of lignin and albumin having energy equal to the difference between the two levels, in other words the natural forces combine with somatic equilibrium in the alimentary canal using HCL, Chyme and water which is covalent, so it will split giving the centrifugal forces of the cell activated by the sodium and potassium pump a jump to effect fluids that have been built up by this Magistral, a product that acts on the brain and body in such a way as to create a quantitative amount of fluid. Dosage to be given can be determined by analysis of the mass and weight of a person, as to bone mass, muscle and existing bacteria in the blood, as this Magistral product brings a fresh supply of blood to the areas, namely the alimentary canal or gut and genitalia to point of ejaculation. Its colors are yellow to orange.

Ejaculation.

Stale Red Clover

Common Names: Clever Grass, Purple Clover, Marl Grass, Cow Grass.

Botanical Name: Trifolium pratense.

### NO.: LEGUMINOSAE

Red Clover is of the Leguminosae family and is one of God's greatest blessings to man. Pg. 301 Back to Eden. It contains silica and other earthy salts which makes it an antidote to cancer. I used this herb because of its nitrogen-fixing bacteria. These bacteria may help the viscid, stringy mucus secreted in the air passages or in the stomach, especially when produced an as morbid discharge. (Observation) One of the four natural humors (the cold and moist) in ancient physiology. {Gr. Phlegm, inflammation, phlegm>phlegm, burn} In sexual health this discharge is indispensable. Therefore, the use of Couch Grass with Stale Red Clover is indispensable, considering, Nitrogen Fixation noun 1. The conversion of atmospheric nitrogen into nitrates by soil bacteria, either free living or in symbiotic relations with the roots of certain leguminous plants. Nitrogen Balance noun The relationship between the nitrogen intake and the nitrogen output of the human body. An excess of intake gives a plus balance; and excess of output gives a minus balance.

Nitro Bacterium noun any of various soil bacteria involved in the process of nitrification, especially the nitrous group (genus Nitrosomonas), which converts ammonia into nitrites and the nitric group (genus Nitrobacter), which oxidizes nitrites into nitrates.

Nitrous Oxide: Laughing Gas.

Nitro Bacterium noun symbol N A nonmetallic element that constitutes nearly four fifths of the air by volume, occurring as a colorless, odorless, almost inert diatomic gas, N₂, in Of the air by volume, occurring a colorless, odorless, almost inert diatomic gas, N₂ in various minerals and in all proteins and used in a wide variety of important medications as nitro-glycerin, a vasodilator.

Red Clover is also very soothing to the nerves. The renals are strengthened because it has the ability to knit back the capillaries and dissolve small calculi. A quote from Herbalists of the past: “The likelihood is that whatever virtue the Red Clover can boast for counteracting a scrofulous disposition and as an antidote to cancer, resides in its highly elaborated lime, silica and other earthy salts.” Pg 233 Indian Herbalogy.

Red Clover promotes healthy granulation. Its colors are white, pink, purple, red, yellow and combinations of shades thereof. A unique quality is hairy stems.

Mugwort

Common Name: Felon Herb.

Botanical Name: Artemisia vulgaris, L.

### N: O.: COMPOSITAE

Medicinal Properties: Mugwort is important in kidney and bladder inflammations. It dissolves kidney stones. I used it because it is soothing to the nerves. It also is good for worms. Culpeper used this herb for counteraction of opium. Pg 201 Ibid. Its colors are yellow and purplish. It is also an emmenagogue. It hastens blood flow. It work on the genitals.

Passion Flower Passiflora incarnata. L.

### O.N.: PASSIFLORACEAE

Passion Flower is a tropical verb having woody climbing tendrilled vines. The flowers are very striking. Spanish explorers called it “Passion Flower” Because it resembled the crown of Christ's halo of thorns. This herb is useful in controlling convulsions, of all kinds and is good for neuralgic pains. Its colors are white, purple and blue.

Saffron

Common Names: Dyers Saffron, Saffron Seed, American Saffron, False Saffron, Thistle Saffron.

Botanical Name: Carthamus tinctorius. Modern: Crocus Sativa

### N.O.: IRIDACEAE

Saffron has laxative properties; a little goes a long way. This herb is good for the nerves and will produce profuse perspiration when taken hot. Its colors are orange to red, mixed with yellow.

Thyme

Common Names: Garden Thyme, Mother of Thyme.

Botanical Name: Thymus vulgaris.

### N.O.: LABIATE

Medicinal Properties: Thyme will increase blood flow. I choose this herb because of its ennenagogue properties. Thyme along with Saffron produces cymene, one of three isometric liquid compounds, C₁₀H₁₄ with lemon like odor, contained in several volatile oils, as cumin, wild thyme, etc, and obtained by distilling camphor with phosphoric anhydride especially para-cy-mene, used as a solvent and in organic synthesis sometimes called camphogen, also cymol{<Ger., Kyminon>Heb. Kammon, cumin}. Its colors are yellow to green. Thyme works on the cerebellum and, Saffron works on the genitals. Canto I Song of Solomon vs. 14: My beloved is unto me as a cluster of camphor in the vineyards of En Gedi. (NIV)

Scull Cap

Common Names: Blue Scull Cap, Blue Pimpernel, Hood-Wort, Mad Dog Weed, Side Flower, Skullcap Helmet Flower, American Scull Cap.

Botanical Name: Scutellaria lateriflora, L.

### N.O.: LABIATE

Bodily Influence: Slightly astringent, nervine, tonic. I used Scull Cap for its ability to control nervous irritation, as it acts on the cerebro spinal centers. Chemically it contains essential oil, yellowish-green fixed oil, a volatile matter, albumen, an astringent principle, lignin, chloride of soda, salts of iron, silica, etc. Its color is pale blue. On the color spectrum blue is in the upper body mainly the head.

The other colors of note are green for the heart, orange and yellow for the kidneys and red for the peritoneal area.

Putting all the colors in perspective, I can conclude that the red ray in nitro-bacteria, which works synergistically with the albumin and the green fixed oil of Skull Cap, which is volatile and causes the nervine properties of Skull Cap to increase; rendering its potential to quiet and strengthen the cerebro spinal centers and cool the body making the patient free from disturbances of all nervous affections. Pgs. 249 & 250. Ibid. I correlated this information with Eucalyptol noun a crystalline compound C₁₀H₁₆-2HCL with camphor like color, derived from eucalyptol also Eucalyptene Hydrochloride.

Eucane noun Either of two important compounds similar in effect to cocaine, but less toxic, used for local anesthesia, known as eucaine a and eucaine b, both compounds are employed as hydrochloric acid salts.

employed as hydrochloric acid salts.

Specialty (Spe sh ul) noun The state of being special or of having peculiarity: distinguishing mark.

Specialist An occupation or study limited to one particular line or practice i.e. Neuromuscular Disease, Neuro-Psychology noun The study of the relationships existing between the mind and the nervous system.—Neuro-Psychological adj.

Tonic a medicine that gradually restores the normal tone of organs from a condition of debility: hence whatever imparts vigor or tone.

Medicine The healing art; the science of the preservation of health and of treating disease for the purpose of a cure specifically, as distinguished form surgery or obstetrics.

Tonicity (To nic ity) noun The state of being tonic, specially, the peculiar elastic condition of healthy tissue hence health and vigor generally.

Please verb ‘plez/-ed/-ing/-s [ME plesen, plaisen, Fr. MF plaisir, Fr. L placere; a ki-n to OE floh cliff., ON flo Layer, L placare to reconcile, placate, GK plak-, plax flat surface, Lith, plakanas flat, and perh. to OE flor floor—more at FLOOR] iv 1: to afford or give pleasure, delight, or agreeable, satisfaction: be agreeable<the chief object of a play should be to please and entertain> 2: [placate, satisfy; specif.: to satisfy sexually 3: to be the will or pleasure of <please God> <May it please your Majesty> Rejoice, may suggest joy marked by enthusiastic or festive happiness<or of even deeper happiness springing from the stirring of those faculties through which man rejoices in knowledge. H. O. Taylor.> GLADDEN, HAPPY, REJOYCE!

Noumenal adj. Of or pertaining to the Noumenon or thing in itself, as opposed to Phenomenal, L. adv. The unknown ground or cause of phenomena, regarded as necessarily assumed by the mind, but the real nature of which is wholly transcendent: the unknowable in it self.

Cutin i zation noun The modification of cell walls by the presence of cutin, making them waterproof to enhance astringent properties.

The Magistral Viagra works because it is made from herbs from God's garden.

Cite₁ Genesis 1: 28 and 29.

Cite₂ Pg. 111, Become Younger By N. W. Walker Dr. of Science, Aurora Book Denver Colo.

Dr. Walker explains how the glands work synergistically to, make the body younger. Spermatocyte noun. a cell giving rise to sperm cells.

1. Viagra is a formula that contains seven natural botanical herbs. It works on the endocrine glands to engorge the penis: to the point of erection.
